(19) United States
(12) Patent Application Publication
(10) Pub. No.: US 2005/0197697 A1
(43) Pub. Date: Sep. 8, 2005

(54) Corrective element for presbyopia

(76) Inventors: Georges Baikoff, Marsinie (FR); Scott Hampton, Cumming, GA (US)

Correspondence Address: SHELDON & MAK, INC, 225 SOUTH LAKE AVENUE, 9TH FLOOR, PASADENA, CA, 91101, US

(21) Appl. No.: 11/053,527

(22) Filed: Feb. 7, 2005

Related U.S. Application Data
(63) Continuation of application No. 10/142,088, filed on Jan. 13, 2003, now abandoned.

(51) Int. Cl. A61F 2/14

(52) U.S. Cl. 623/4.1

(57) ABSTRACT

A corrective element useful in the treatment of presbyopia has an hourglass shape.

### BACKGROUND OF THE INVENTION

Corrective elements useful in the treatment of presbyopia are well-known in the industry. For example, U.S. Pat. No. 5,465,737 and French Patent Document 98 12384 describe such corrective elements for presbyopia and describe their implementation.

### DRAWINGS

These and other features, aspects and advantages of the present invention will become better understood with reference to the following description, appended claims and accompanying drawings where:

FIG. 1 is a top view of a corrective element for presbyopia having features of the invention;

FIG. 2 is a side view of the corrective element illustrated in FIG. 1;

FIG. 3 is an end view of the corrective element illustrated in FIG. 1;

FIG. 4 is an alternative end view of the corrective element illustrated in FIG. 1; and

FIG. 5 is a perspective view of a second corrective element having features of the invention.

### DETAILED DESCRIPTION

The following discussion describes in detail one embodiment of the invention and several variations of that embodiment. This discussion should not be construed, however, as limiting the invention to those particular embodiments. Practitioners skilled in the art will recognize numerous other embodiments as well.

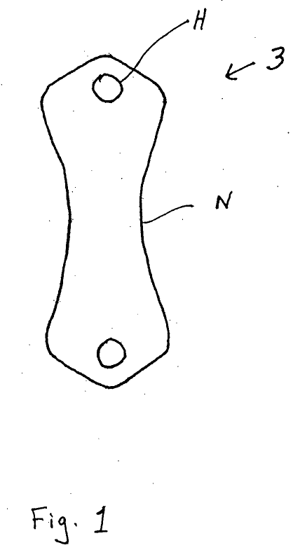

The invention is a corrective element for presbyopia 3 having a unique hourglass shape. FIG. 1 illustrates a typical embodiment of the invention having an arched segment and an hourglass design. In this embodiment, when viewed in a top view, the center portion of the element 3 has a narrower dimension N than the element at either end. This design assists in maintaining the position of the element after implantation.

The center portion of element 3 has arcuate inner and outer surfaces having radii of curvature.

Optionally, a hole or indentation H at either or both ends of element 3 may be provided to allow for greater control in handling the element 3 upon implantation and may further serve as an egress for suturing the element 3 in the eye. Such an optional hole H is illustrated in FIG. 1 at both ends of element 3.

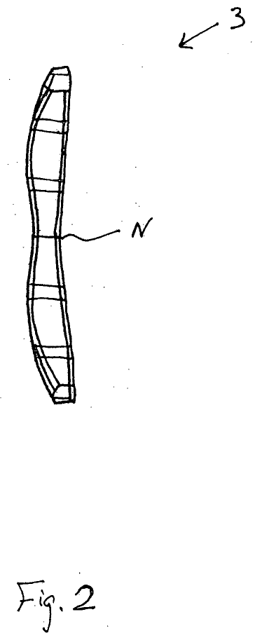

FIG. 2 illustrates a side view of the embodiment illustrated in FIG. 1 having a further non-limiting enhancement whereby the center portion of the element 3 has a narrower dimension at the center of the element 3 in an hourglass design. Even with a tapered center portion, the inner and outer surfaces of the element possess radii of curvature.

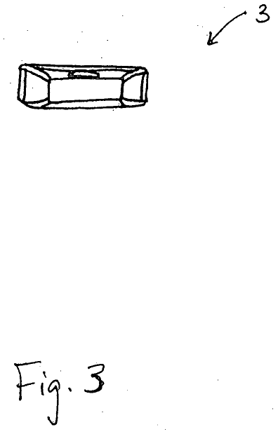

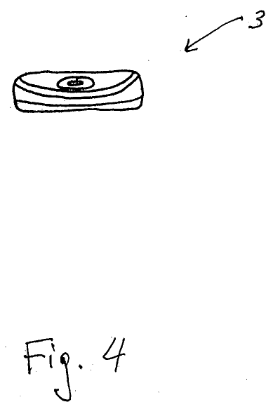

FIGS. 3 and 4 each illustrate an end view of non-limiting embodiments of the invention, wherein the end has either a squared or rounded design. These non-limiting embodiments are illustrative of other contemplated designs wherein the end of the element is designed in each way as to avoid injury when inserted into position within the sclera of the eye. In both of these illustrations, an optional hole or indentation is depicted.

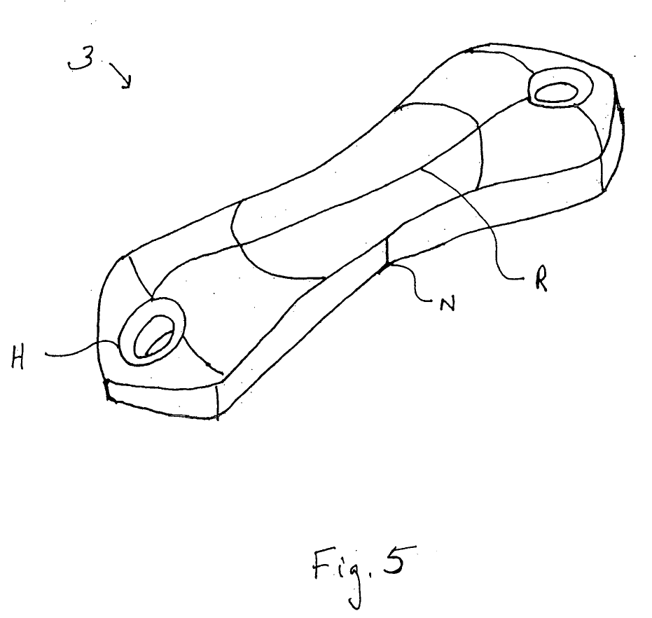

FIG. 5 is a non-limiting preferred embodiment of the element having an hourglass shape. As can be seen by the change of curvature, the element is narrower in dimension at the center of the element and greater in dimension at the ends of the element. The element optionally possesses one or more ridges on the surface of the element. As illustrated in FIG. 5, a ridge R runs along the center of the element. Such a ridge also provides for a greater case of handling of the element upon implementation of the device in the sclera of the eye.

Having thus described the invention, it should be apparent that numerous structural modifications and adaptations may be resorted to without departing from the scope and fair meaning of the instant invention as set forth hereinabove and as described hereinbelow by the claims.

1. An element useful in the correction of presbyopia and suitable for being implanted in the eye opposite the ciliary body, the element having an hourglass design.

2. The element of claim 1 wherein the element has a hole at the end of the element.

3. The element of claim 1 wherein the element has a ridge on the surface of the element.
